(19) United States
(12) Patent Application Publication
(10) Pub. No.: US 2005/0054586 A1
(43) Pub. Date: Mar. 10, 2005

(54) Treatment of ophthalmic disorders

(76) Inventors: Stephen P. Bartels, Pittsford, NY (US); Sebastiano Mangiafico, Solarino (IT)

Correspondence Address: Bausch & Lomb Incorporated, One Bausch & Lomb Place, Rochester, NY, 14604-2701, US

(21) Appl. No.: 10/876,014

(22) Filed: Jun. 24, 2004

Related U.S. Application Data
(60) Provisional application No. 60/483,704, filed on Jun. 30, 2003.

(51) Int. Cl. A61K 31/7048; A61K 31/452; A61K 31/4025; A61K 31/366

(52) U.S. Cl. 514/27; 514/320; 514/422; 514/457

(57) ABSTRACT

Pharmaceutical compositions comprising coumarin bases and salts thereof are useful for treat various ophthalmic disorders. The active ingredient includes cloricromene or its hydrochloride salt.

### FIELD OF INVENTION

This invention relates to pharmaceutical compositions comprising coumarin bases and salts thereof to treat various ophthalmic disorders.

### BACKGROUND OF THE INVENTION

Coumarins include a class of phenol substances characterized by fused benzene and α-pyrone rings. Cloricromene and carbocromene belong to the coumarin family and are represented by the formulae:

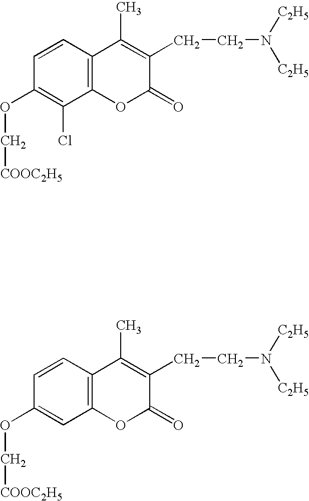

U.S. Pat. No. 4,452,811 (della Valle) discloses that carbocromene and cloricromene have vasodilatory activity and may used to treat coronary diseases caused by the obstruction of blood vessels. U.S. Pat. No. 4,349,566 (della Valle) discloses that cloricromene exhibits antiarrhythmic activity. U.S. Pat. No. 4,362,741 (della Valle) discloses that cloricromene may be used to prevent aggregation of platelets. WO 2000/76498 discloses cholesterol-lowering activity of cloricromene and other coumanins. WO 2002/10148 discloses various coumarin derivatives for treating major pathologies such as peripheral ischaemia and organ ischaemia, electrical alterations of the myocardium and other organs resulting from the release of pro-inflammatory molecules, peripheral and cerebral vasculopathies, as well as additional pathologies.

### SUMMARY OF THE INVENTION

This invention provides methods of treating ophthalmic disorders, comprising administering to a subject a coumarin base or a salt thereof, and especially a compound of the formula (I) or a pharmaceutically acceptable salt thereof: 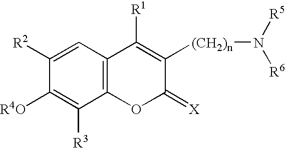
wherein:

X is O or S;

n is zero of an integer of 1 to 10;

R¹ is methyl or phenyl;

R² and R³ are independently H, OH, allyl, halogen or methyl;

R⁵ and R⁶ are independently hydrogen, a C₁-C₄ alkyl group, or R⁵ and R⁶ together with the nitrogen atom form a N-heteroring optionally containing other heteroatoms; and

R⁴ is H; C₁-C₁₀ alkyl or alkenyl optionally substituted with one or more ether, thioether, ester or amino radicals and optionally substituted with OH, amido, sugar residues or amino acid residues; or a radical of formula (II):

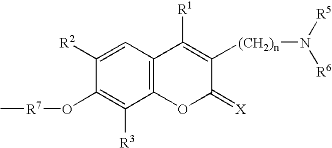

R⁷ is a C₁-C₁₀ alkylene chain optionally substituted with one or more ether, thioether, ester or amino radicals and optionally substituted with OH, amido, sugar residues or amino acid residues.

The compound or salt thereof may be administered orally or by injection, or delivered via a sustained release device implanted or injected in eye tissue, such as the back of the eye.

Ophthalmic disorders include diabetic retinopathy, diabetic macular edema, retinal vascular occlusive disease, uveitis, and choroiditis.

One class of compounds include compounds of formula (III), or a pharmaceutically acceptable salt thereof, containing 8-chloro or 8-bromo substitution: 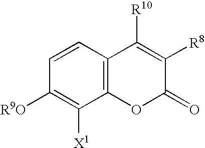
wherein:

R⁸ is an alkyl group having a basic substituent;

R⁹ is an alkyl group substituted with a basic group, an alkenyl group, a carboxy alkyl group or an alkoxy carbonyl alkyl group;

R¹⁰ is hydrogen, alkyl or aryl; and

X′ is chlorine or bromine.

One preferred compound is cloricromene, especially the hydrochloride salt thereof.

The methods of this invention specifically include, for mammals including humans:

treatment of diabetic retinopathy;

prevention of retinal hemorrhaging;

prevention of visual acuity loss in a subject with an ophthalmic disorder;

reducing hard exudates in eye tissue; and

delaying progression of retinal damage, especially in diabetic subjects.

### DETAILED DESCRIPTION

The pharmaceutical compositions of this invention comprise a coumarin base or pharmaceutically acceptable salt thereof. These compounds include compounds of the formula (I): 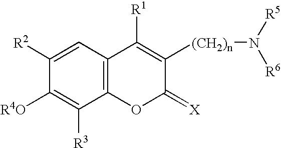
wherein:

X is O or S;

n is zero of an integer of 1 to 10;

R¹ is methyl or phenyl;

R² and R³ are independently H, OH, allyl, halogen or methyl;

R⁵ and R⁶ are independently hydrogen, a C₁-C₄ alkyl group, or R⁵ and R⁶ together with the nitrogen atom form a N-heteroring optionally containing other heteroatoms; and

R⁴ is H; C₁-C₁₀ alkyl or alkenyl optionally substituted with one or more ether, thioether, ester or amino radicals and optionally substituted with OH, amido, sugar residues or amino acid residues; or a radical of formula (II):

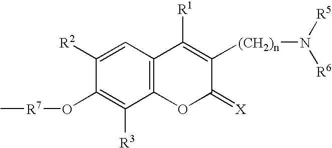

R⁷ is a C₁-C₁₀ alkylene chain optionally substituted with one or more ether, thioether, ester or amino radicals and optionally substituted with OH, amido, sugar residues or amino acid residues.

Representative —OR⁴ radicals include hydroxyl, ethyoxcarbonylmethoxy, 2-hydroxyhexyloxy, propyloxy, and 2-hydroxyopropyloxy.

Representative radicals composing the —(CH₂)ₙ—N(R⁵)(R⁶) moiety include piperidino ethyl, morpholino ethyl, diethylamino ethyl or diethylamino propyl.

A preferred salt is the hydrochloride salt.

Various coumarins and the preparation thereof are disclosed in the following literature, the disclosures of which are incorporated herein by reference: U.S. Pat. No. 4,452,811 (della Valle); U.S. Pat. No. 4,349,566 (della Valle); U.S. Pat. No. 4,362,741 (della Valle); WO 2000/76498; WO 2002/10148; and U.S. Pat. No. 4,296,039. All the compounds used in this invention may be prepared by methods known in the art.

Preferred compounds specifically include 8-bromo or 8-chloro derivatives of the formula (III): 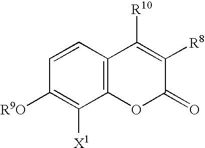
wherein:

R⁸ is an alkyl group having a basic substituent, such as piperidino ethyl, morpholino ethyl, diethylamino ethyl or diethylamino propyl;

R⁹ is an alkyl group substituted with a basic group, an alkenyl group, a carboxy alkyl group or an alkoxy carbonyl alkyl group;

R¹⁰ is hydrogen, alkyl or aryl; and

X′ is chlorine or bromine.

A preferred compound is cloricromene or its hydrochloride salt. 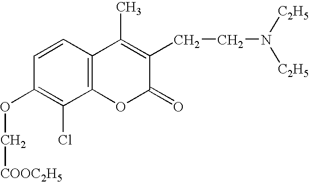
The hydrochloride salt of cloricromene is also known under the tradename Proendotel and may be prepared by the process described in U.S. Pat. Nos. 4,296,039 and 4,452,811.

It has been found that these compounds and salts may be administered to mammals, including humans, to treat various ophthalmic disorders or pathologies. Such disorders include diabetic retinopathy, diabetic macular edema, retinal vascular occlusive disease, uveitis (including posterior segment uveitis and anterior segment uveitis), and choroiditis.

The compounds or salts thereof may be administered orally to a subject in need to treatment. Oral preparations may have the form of dragees, tablets or capsules such as gelatin capsules. Generally, the active is combined with conventional pharmaceutical excipients, carriers or diluents including water, vegetable oils, gum arabic, gelatin, cellulose derivatives, polyglycols and/or emulsifying agents.

The compounds or salts thereof may be administered by injection, including intramuscularly or intravenously. Generally, the active is combined with conventional pharmaceutical excipients, carriers or diluents such as water or saline solution. Additionally, the injectable preparations may be administered locally by injecting the preparation directly into eye tissue.

The compounds or salts may be contained in a sustained release device, wherein the device is implanted or injected in the body to release the: active over time. Preferably, the device is implanted or injected in eye tissue. Examples of such devices are found in the following patents, the disclosures of which are incorporated herein by reference: U.S. 2002/0086051A1 (Viscasillas); U.S. 2002/0106395A1 (Brubaker); U.S. 2002/0110591A1 (Brubaker et al.); U.S. 2002/0110592A1 (Brubaker et al.); U.S. 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); and U.S. Pat. No. 6,375,972 (Guo et al.).

Pharmaceutical preparations will contain a pharmaceutically effective amount of the compound or its salt. Generally, the preparations contain the active in an amount of 10 to 500 mg. Generally, the compound or its salt is administered in a daily dosage of 10 to 500 mg, more specifically, a daily dosage of 25 to 200 mg, and most preferably, 50 to 200 mg.

Pharmaceutical preparations containing the pharmaceutically effective amount of the compound or its salt may further contain other actives, especially when the compound or its salt is included in an implantable sustained release device. Examples of such supplemental active agents include: anesthetics and pain killing agents such as lidocaine and related compounds and benzodiazepam and related compounds; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility impending agents such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydride inhibitors; diagnostic agents; antiapoptosis agents; gene therapy agents; sequestering agents; reductants such as glutathione; antipermeability agents; antisense compounds; antiproliferative agents; antibody conjugates; antidepressants; bloodflow enhancers; antiasthmatic drugs; antiparasiticagents; non-steroidal anti inflammatory agents such as ibuprofen; nutrients and vitamins: enzyme inhibitors: antioxidants; anticataract drugs; aldose reductase inhibitors; cytoprotectants; cytokines, cytokine inhibitors, and cytokin protectants; uv blockers; mast cell stabilizers; and anti neovascular agents such as antiangiogenic agents like matrix metalloprotease inhibitors.

A clinical study was conducted in order to test the safety and efficacy of the compounds for treating ophthalmic disorders. The study included 40 human patients with type-1 diabetes and affected by non-proliferative diabetic retinopathy. Twenty of the patients received one tablet daily containing cloricromene hydrochloride (100 mg), whereas twenty of the patients formed a control group and received no treatment. Patients were randomly assigned either to receive cloricromene or to receive no treatment. The patients included males and females over 45 years of age assessed with type-1 diabetes mellitus and non-proliferative retinopathy assessed by fundus photography and fluoroscein angiography. For patients with bilateral disease, both eyes were evaluated. For patients with unilateral disease, the affected eye served as the study eye. Excluded from the study were subjects: affected with proliferative diabetic retinopathy; having visual acuity less than 2/10; with a history of renal failure; or receiving treatment with anti-coagulants, platelet anti-aggregants, or fibrinolytics.

The results summarized in the following tables are based on start of study versus one-year study period. Visual acuity was assessed by the patients' use of an eye chart. The presence of hemorrhaging, hard exudates or vascular leakage in the retina was evaluated as a means of grading degree of retinal lesion. Hemorrhaging and hard exudates were assessed primarily by observing the stereoscopic color fundus photographs of the retain. Vascular leakage was assessed primarily by fluorescein staining.

Visual Acuity

| | Cloricromene | Control |
|---|---|---|
| Improved | 11 (55%) | 2 (10%) |
| Stable | 8 (40%) | 11 (55%) |
| Worse | 1 (5%) | 7 (35%) |
| Total | 20 (100%) | 20 (100%) |

Hard Exudates

| | Cloricromene | Control |
|---|---|---|
| Improved | 14 (70%) | 5 (20%) |
| Stable | 6 (30%) | 8 (40%) |
| Worse | 0 (0%) | 7 (0%) |
| Total | 20 (100%) | 20 (100%) |

Retinal Hemorrhages

| | Cloricromene | Control |
|---|---|---|
| Improved | 13 (65%) | 3 (15%) |
| Stable | 6 (30%) | 7 (35%) |
| Worse | 1 (5%) | 10 (50%) |
| Total | 20 (100%) | 20 (100%) |

Vascular Leakage

| | Cloricromene | Control |
|---|---|---|
| Improved | 3 (15%) | 1 (5%) |
| Stable | 16 (80%) | 11 (55%) |
| Worse | 1 (5%) | 8 (40%) |
| Total | 20 (100%) | 20 (100%) |

These clinical results demonstrate that cloricromene hydrochloride was effective in delaying the progression of retinal damage in diabetic patients. Accordingly, more invasive treatments at later states of the disease can be avoided. In comparison to Controls, the tested formulations prevented retinal hemorrhaging, prevented visual acuity loss and reduced formation of hard exudates in eye tissue.

Although various preferred or illustrative embodiments have been described, a person of ordinary skill in the art will readily appreciate variations of such described embodiments.

1. A method of treating diabetic-related ophthalmic disorders, comprising administering to a diabetic subject an effective amount of a compound of the formula (I), or a pharmaceutically acceptable salt thereof: 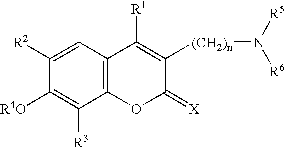
wherein:
X is O or S;
n is zero of an integer of 1 to 10;
R¹ is methyl or phenyl;
R² and R³ are independently H, OH, allyl, halogen or methyl;
R⁵ and R⁶ are independently hydrogen, a C₁-C₄ alkyl group, or R⁵ and R⁶ together with the nitrogen atom form a N-heteroring optionally containing other heteroatoms; and
R⁴ is H; C₁-C₁₀ alkyl or alkenyl optionally substituted with one or more ether, thioether, ester or amino radicals and optionally substituted with OH, amido, sugar residues or amino acid residues; or a radical of formula (II): 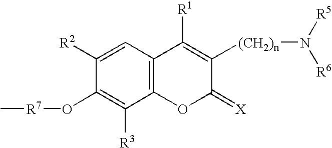
R⁷ is a C₁-C₁₀ alkylene chain optionally substituted with one or more ether, thioether, ester or amino radicals and optionally substituted with OH, amido, sugar residues or amino acid residues
wherein visual acuity of said subject is improved.

2. The method according to claim 1, wherein said compound is cloricromene or a salt thereof.

3. The method according to claim 2, wherein said compound or salt thereof is administered orally.

4. The method according to claim 3, wherein said compound or salt thereof is contained in a tablet or capsule further comprising a pharmaceutically acceptable carrier.

5. The method according to claim 2, wherein said compound or salt thereof is injected in eye tissue.

6. (canceled)

7. The method according to claim 1, wherein said compound or salt thereof is contained in a sustained release device, said method further comprising implanting said device in eye tissue.

8. The method according to claim 7, wherein said device is implanted in the back of the eye.

9. The method according to claim 1, wherein said compound or salt thereof is administered in a dosage of 25 to 500 mg.

10. The method according to claim 9, wherein said compound or salt thereof is administered in a dosage of 50 to 200 mg.

11. The method according to claim 3, wherein said compound or salt thereof is administered in a daily dosage of 50 to 200 mg.

12. The method according to claim 11, wherein said compound or salt thereof is administered in a daily dosage of about 100 mg.

13. The method according to claim 2, wherein said ophthalmic disorder is selected from the group consisting of diabetic retinopathy and, diabetic macular edema.

14-18. (canceled)

19. The method according to claim 2, comprising administering the hydrochloride salt of said compound to treat diabetic retinopathy.

20-23. (canceled)

24. A method of preventing retinal hemorrhaging, comprising administering to a subject cloricromene or a pharmaceutically acceptable salt thereof in a daily dosage of 25 to 500 mg.

25. (canceled)

26. A method of preventing visual acuity loss in a diabetic subject, comprising administering orally to the subject cloricromene or a pharmaceutically acceptable salt thereof in a daily dosage of 25 to 500 mg.

27. (canceled)

28. A method of reducing hard exudates in eye tissue, comprising administering orally to a subject cloricromene or a pharmaceutically acceptable salt thereof in a daily dosage of 25 to 500 mg.

29. (canceled)

30. A method of delaying progression of retinal damage in a diabetic subject, comprising administering to the subject cloricromene or a pharmaceutically acceptable salt thereof.

31-32. (canceled)

33. The method according to claim 2, comprising administering the cloricromene or salt thereof for a period of at least one year.

34. The method according to claim 24, comprising administering the cloricromene or salt thereof for a period of at least one year.

35. The method according to claim 24, wherein the cloricromene or salt thereof is administered to a diabetic patient.

36. The method according to claim 28, comprising administering the cloricromene or salt thereof for a period of at least one year.

37. The method according to claim 28, wherein the cloricromene or salt thereof is administered to a diabetic patient.
